(19) United States
(12) Patent Application Publication
(10) Pub. No.: US 2026/0061115 A1
(43) Pub. Date: Mar. 5, 2026

(54) SELECTIVE MEDICAMENT DELIVERY ASSEMBLY WITH FLUIDIC ACTIVATION

(71) Applicant: SHL Medical AG, Zug (CH)

(72) Inventors: Martin Michael Coyne, III, Boonton, NJ (US); Richard James Caizza, Union City, NJ (US)

(21) Appl. No.: 19/107,175

(22) PCT Filed: Sep. 1, 2023
(86) PCT No.: PCT/EP2023/072541

§ 371 Date: Feb. 27, 2025

Related U.S. Application Data
(60) Provisional application No. 63/402,547, filed on Aug. 31, 2022.
(60) Provisional application No. 63/444,597, filed on Feb. 10, 2023.

(30) Foreign Application Priority Data
Sep. 30, 2022 (EP) 22199289.4

(51) Int. Cl. A61M 5/14; A61M 5/142; A61M 5/158

(52) U.S. Cl. CPC A61M 5/1409; A61M 5/142; A61M 2005/1585; A61M 2005/1588

(57) ABSTRACT

The present disclosure provides a sub-assembly for a medicament delivery device, comprising: a housing, a closed chamber, and a tubing set transversally connected to the chamber. The tubing set is arranged to provide one of a first medicament flow and a second medicament flow, a first fluid pressure is effectuated in the chamber when the first medicament is provided and a second fluid pressure having a different value from the first fluid pressure is effectuated when the second medicament is provided, thereby a fluid pressure change is effectuate due to a change between the provision of the first medicament and the second medicament. The sub-assembly further includes a shuttle arranged in the chamber and connected to the tubing set. The shuttle is configured to move transversally between a first position and a second position within the chamber when the fluid pressure change within the chamber exceeds a predetermined level.

### CROSS-REFERENCE TO RELATED APPLICATIONS

The present application is a U.S. National Phase Application pursuant to 35 U.S.C. § 371 of International Application No. PCT/EP2023/072541 filed Aug. 16, 2023, which claims priority to (i) U.S. Provisional Application No. 63/402,547 filed Aug. 31, 2022, (ii) EP Patent Application No. 22199289.4 filed Sep. 30, 2022, and (iii) U.S. Provisional Application No. 63/444,597 filed Feb. 10, 2023. The entire disclosure contents of these applications are herewith incorporated by reference into the present application.

### TECHNICAL FIELD

The present invention relates to a medicament delivery device, in particular to a selective medicament delivery assembly with fluidic activation.

### BACKGROUND

Many devices for administering a medicament are designed with a number of functions for providing ease of use for medicament administration. The functions that are applicable comprise mixing of the medicament with a diluent, if the medicament is in powder form, priming of the medicament container, thereby reducing any air trapped therein, penetration of an injection needle into the tissue of a patient on an injection site, injection of a dose of medicament at the injection site, withdrawal of the injection needle after an injection sequence is ended and covering of the injection needle after use of the medicament delivery device.

Some medicament delivery devices require that a delivery member protrudes a certain length from the proximal end of the device when expelling a medicament through the delivery member. When using injection devices, some types of medicament require that the dose is injected at a certain depth into the tissue of the patient. Depending on the type of medicament and/or treatment, the injection depth could be any of intradermal, subcutaneous and intramuscular. In that respect, the injection depth also depends on the condition of the patient, i.e. the amount of fat in the subcutaneous tissue or depth of the muscular tissue relative to the skin and subcutaneous tissue. Because of the demands on the depth of the injection, devices have been developed to be arranged with mechanisms that can adjust the insertion depth of a needle. For instance, subcutaneous SC infusion for adults typically requires 4-6 mm insertion, while intramuscular IM injection typically requires ≥12.7 mm insertion.

Some prior art has disclosed meeting the depth requirements by selecting discrete needles with different fixed lengths corresponding to the desired route of administration prior to insertion. However, numerous discrete needles may be impractical or infeasible.

Some other prior art has disclosed needles with adjustable lengths protruding from the proximal end, the length protruding from the proximal end is adjusted by electrical means, such as an electrical circuit arrangement or a pum driven by a motor. Such types of devices require complicated components and structures, which is not cost effective. Improved solutions are thus needed to provide greater flexibility around needle insertion, to automate needle insertion as certain aspects of an infusion of medicament take place, and to simplify needle insertion for users of medicament delivery devices.

### SUMMARY

A general object of the present disclosure is to provide a sub-assembly for a medicament delivery device that solves or at least mitigates the problems of the prior art.

There is hence according to an aspect of the present disclosure provided a sub-assembly for a medicament delivery device, comprising: a housing; a closed chamber within the housing; a tubing set transversally connected to the housing, wherein the tubing set is arranged to provide a medicament flow, a fluid pressure is effectuated in the chamber when the medicament is provided; a shuttle arranged in the chamber and connected to the tubing set, wherein the shuttle is configured to move transversally between a first position and a second position within the chamber when the fluid pressure exceeds a predetermined level, such that an administration path in the chamber is opened for delivery of the medicament.

According to one embodiment, the tubing set is arranged to provide one of a first medicament flow and a second medicament flow, a first fluid pressure is effectuated in the chamber when the first medicament is provided and a second fluid pressure having a different value from the first fluid pressure is effectuated in the chamber when the second medicament is provided, thereby a fluid pressure change is effectuate due to a change between the provision of the first medicament and the second medicament.

According to one embodiment, the shuttle is configured to move transversally between a first position and a second position within the chamber when the fluid pressure change exceeds a predetermined level, such that a first administration path or a second administration path in the chamber is opened for selectively delivery of the first medicament and the second medicament.

According to one embodiment, a needle connected to the administration path and arranged in the housing, wherein the needle is arranged to be longitudinally movable in respect to the housing when the shuttle is move transversally from the first position to the second position.

According to one embodiment, the sub-assembly for a medicament delivery device further comprises the needle is connected to the first administration path and the second administration path and arranged in the housing, wherein the needle is arranged to be longitudinally movable in respect to the housing between a retracted position and an extended position such that a length of the needle extending outside the housing is adjustable, wherein the length of the needle extending outside the housing when the needle is in the retracted position is shorter than the length of needle extending outside the housing when the needle is in the extended position.

According to another embodiment, the sub-assembly for a medicament delivery device further comprises an elastic element longitudinally arranged along the needle and attached to the housing at one end and attached to a needle hub at another end, wherein the needle hub is attached to the needle and longitudinally movable in respect to the housing such that the tension of the elastic element is changed and the length of the needle extending outside the housing is adjusted, and a needle retention bar arranged to releasably engage with the needle hub when the needle is in the retracted position, and the needle retention bar is further arranged to transversally shift between an inner position and an outer position due to the transverse movement of the shuttle such that the needle moves longitudinally in respect to the housing.

According to another embodiment, the sub-assembly for a medicament delivery device further comprises the needle retention bar is engaged with the needle hub when the needle is in the retracted position, and the needle retention bar is further arranged to transversally shift between an inner position and an outer position due to the transverse movement of the shuttle such that the needle moves longitudinally from the retracted position to the extended position.

According to another embodiment, the needle is arranged to extend outside the housing from the retracted position to the extended position when the second fluid pressure is effectuated is the chamber by providing the second medicament in the tubing set, wherein the second fluid pressure is higher than the first fluid pressure.

According to another embodiment, the shuttle is configured to move from the first position to the second position and correspondingly, the needle retention bar is configured to shift from the inner position to the outer position, thereby the tension of the elastic element changes and the needle is configured to move from the retracted position to the extended position.

According to another embodiment, the needle is arranged to be retracted from the extended position to a second retracted position when a lower fluid pressure is effectuated in the chamber than the second fluid pressure, wherein the second retracted position is between the retracted position and the extended position.

According to another embodiment, the tubing set comprises a first lumen tube for the first medicament flow, and a second lumen tube for the second medicament flow. In some embodiments, the second lumen tube has a smaller diameter than the first lumen tube.

According to another embodiment, the shuttle comprises a first opening for providing the first medicament from the tubing set to the first administration path and a second opening for providing the second medicament from the tubing set to the second administration path, wherein a transverse distance between the first opening and the second opening is shorter than the distance between the first administration path and the second administration path thereby the first administration path is opened and the second administration path is blocked when the shuttle is in the first position and the second administration path is opened and the first administration path is blocked when the shuttle is in the second position such that the first medicament or the second medicament is selectively provided via the transverse movement of the shuttle.

According to another embodiment, the needle retention bar is integrated with the shuttle or the needle retention bar is a separate component attached to the shuttle.

According to another embodiment, the needle retention bar comprises a support surface enabling the needle retention bar to be engaged with the needle hub at the inner position and the needle retention bar is arranged to be disengaged with the needle hub at the outer position.

According to another embodiment, the needle retention bar comprises a curved support surface; and wherein the needle hub is configured to move along the curved support surface from the inner position to the outer position.

According to another embodiment, the needle retention bar comprises multiple support surfaces offset to one another in the longitudinal direction.

According to another embodiment, the sub-assembly further comprises an indicator configured to indicate a current administration path being used. In some embodiments, the indicator is a visible symbol configured to be exposed only when the second administration path is used.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

In the present disclosure, when the term “rear direction” is used, this refers to the direction pointing towards the fluid source of the medicament delivery device. When the term “rear part/end” is used, this refers to the part/end of the delivery device, or the parts/ends of the members thereof. Correspondingly, when the term “front direction” is used, this refers to the direction pointing away from the fluid source of the medicament delivery device. When the term “proximal part/end” is used, this refers to the part/end of the delivery device, or the parts/ends of the members thereof.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally”refer to rotation relative to the axis.

Generally, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 schematically shows an example of a front view of a sub-assembly for a medicament delivery device;

FIG. 2 schematically shows an example of a rear view of the sub-assembly for the medicament delivery device;

FIG. 3 schematically shows an example of an overview of the sub-assembly for the medicament delivery device when a first medicament flow is provided in a first lumen tube;

FIG. 4 schematically shows an example of a cross sectional view of the sub-assembly for the medicament delivery device when a first medicament flow is delivered via a first administration path;

FIG. 5 schematically shows an example of an overview of the sub-assembly for the medicament delivery device when the medicament provided in the tubing set changes from the first medicament to a second medicament;

FIG. 6 schematically shows an example of an overview of the sub-assembly for the medicament delivery device when the needle of the sub-assembly moves from a retracted position to an extended position;

FIG. 7 schematically shows an example of a cross sectional view of the sub-assembly for the medicament delivery device when the second medicament flow is delivered via a second administration path.

FIGS. 8-12 schematically show a second embodiment of the invention.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like elements throughout the description.

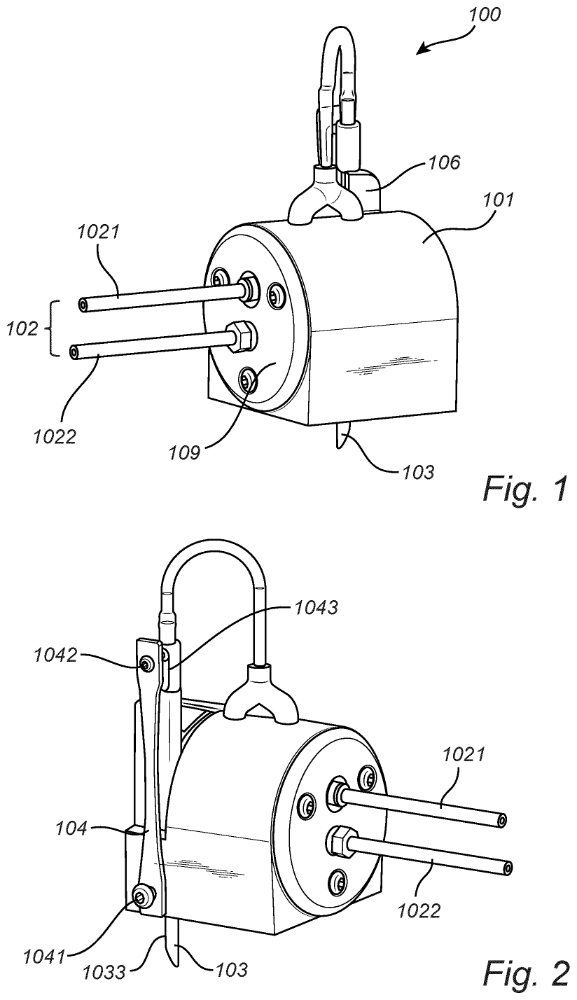

FIGS. 1-7 show a sub-assembly of the first embodiment of the invention. In this embodiment, the sub-assembly is configured to deliver one or more medicaments to different sites via different administration paths; in particular, to different sites that are in different depth under skin of a user. The sub-assembly of the first embodiment will be explained in detail with FIGS. 1-7 below, with two administration paths being used, and a needle 103 is already penetrated the skin when the needle 103 is in a ‘retracted position’ as an example. In other words, how the needle initially penetrates through and therefore positions under the skin are not shown in FIGS. 1-7. All FIGS. 1-7 show that the needle is already penetrated through and therefore positioned under the skin. It should be noted that the sub-assembly is also feasible to provide more than two administration paths targeting to deliver the more than two different sites that are in different depth under skin of a user.

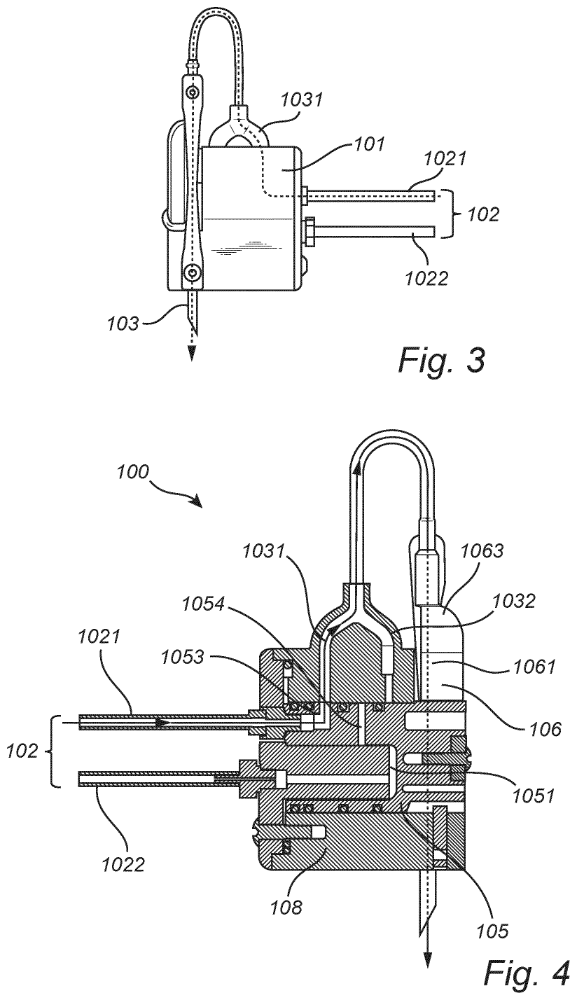

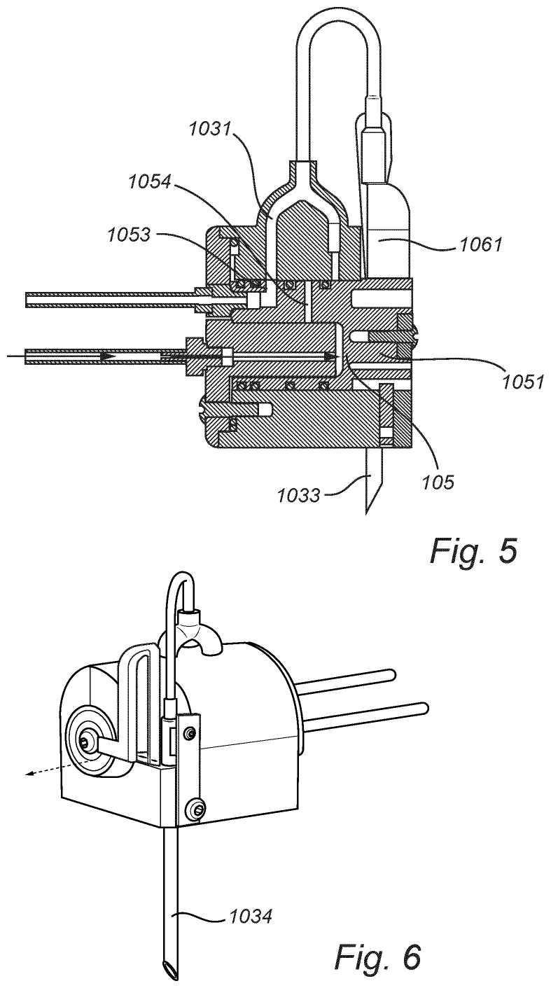

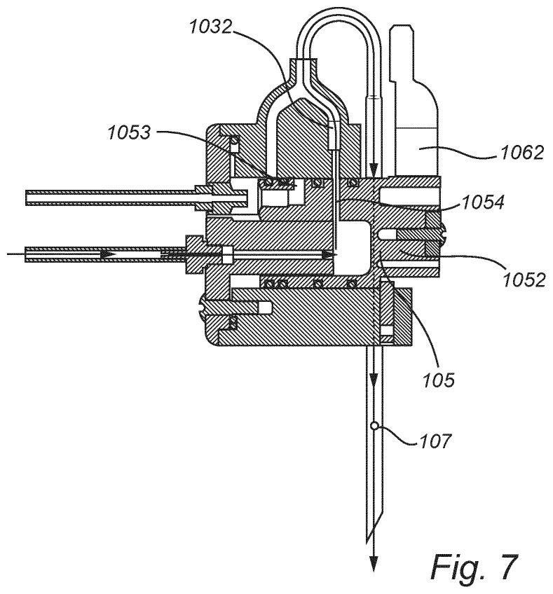

FIG. 1 schematically shows an example of a front view of a sub-assembly for a medicament delivery device. To explain the structure of the sub-assembly for the medicament delivery device in detail, the following content is elaborated in combination with FIG. 2 showing an example of a rear view of the sub-assembly for the medicament delivery device, FIG. 3 showing an example of an overview of the sub-assembly for the medicament delivery device when a first medicament flow is provided in a first lumen tube, FIG. 4 showing an example of a cross sectional view of the sub-assembly for the medicament delivery device when a first medicament flow is delivered via a first administration path, FIG. 5 showing an example of an overview of the sub-assembly for the medicament delivery device when the medicament provided in the tubing set changes from the first medicament to a second medicament, FIG. 6 showing an example of an overview of the sub-assembly for the medicament delivery device when the needle 103 of the sub-assembly moves from the retracted position to an extended position and FIG. 7 showing an example of a cross sectional view of the sub-assembly for the medicament delivery device when the second medicament flow is delivered via a second administration path. The sub-assembly 100 comprises a housing 101, a closed chamber 108 within the housing 101 and a tubing set 102 for providing the first medicament flow or the second medicament flow. When the first medicament is provided in the tubing set 102, as shown in FIGS. 3 and 4, a first fluid pressure is effectuated in the chamber 108 and a second fluid pressure is effectuated in the chamber 108 when the second medicament is provided in the tubing set 102, as shown in FIGS. 5, 6 and 7, the second fluid pressure has a different value from the first fluid pressure, thereby a fluid pressure change is generated due to the change of the provision of the first medicament and the second medicament. According to some embodiments, the first medicament is provided in a non-emergency context and the second medicament is provided in an emergency context. According to some other embodiments, the first medicament is subcutaneous (SC) administration, and the second medicament is intramuscular (IM) administration. It should be noted that, alternatively, the first medicament can be the same medicament as the second medicament. For example, they can be both for subcutaneous (SC) administration but in different depth, e.g., one is delivered to a site that is 6 mm under skin and the other is delivered to a site that is 8 mm under skin. In this example, a better medicament absorption might be the benefit.

According to some embodiments, the tubing set 102 comprises a first lumen tube 1021 and a second lumen tube 1022. According to some other embodiments, the tubing set 102 is a dual lumen tubing comprising the first lumen tube 1021 and the second lumen tube 1022. The first lumen tube 1021 is arranged for the provision of the first medicament and the second lumen tube 1022 is arranged for the provision of the second medicament. The tubing set 102 is fluidly connected to the housing 101. In a preferred example, the tubing set 102 is arranged to be transversally connectable to the housing 101 of the sub-assembly 100. According to some embodiments, the tubing set 102 is integrated with the housing 101 of the sub-assembly 100. According to some other embodiments, the tubing set 102 is separate from the housing 101 of the sub-assembly 100 and is connectable to a fluidic connector of the sub-assembly 100. On one hand, this is advantageous for manufacturing of the sub-assembly 100. On the other hand, this arrangement is also adaptive to a fluid reservoir having an integrated tubing set, the sub-assembly 100 is connected to the tubing set 102 of the fluid reservoir via the fluidic connector when the reservoir is filled with medicament fluid. In some embodiments, in order to rapidly trigger the adjustment of the needle length extending outside the housing 101, the second lumen tube 1022 is arranged with a smaller diameter relative to the first lumen tube 1021, thereby providing higher fluid pressure. This arrangement also makes the sub-assembly 100 have a compact size.

The sub-assembly 100 further comprises a shuttle 105 arranged in the chamber 108 and is connected to the tubing set 102, which is shown in FIGS. 4, 5 and 7. The shuttle 105 is arranged to move transversally between a first position 1051 and a second position 1052 within the chamber 108. The shuttle 105 is further sealably arranged in the chamber 108 and is arranged at an initial position by default. According to some embodiments, the initial position is the first position 1051. The shuttle 105 is further arranged to move to the second position 1052 when the fluid pressure change within the chamber 108 exceeds a predetermined level, such that the first administration path 1031 or the second administration path 1032 in the chamber 108 is opened for selectively delivery of the first medicament and the second medicament. Thus the sub-assembly 100 is activated solely through fluidic pressure without any reliance on a controller and electrical signaling, which greatly reduces the complexity of the sub-assembly. According to some embodiments, the predetermined level is a preset value. According to some other embodiments, the predetermined level is manually configurable. Further according to some embodiments, an end cap 109 is arranged to seal off the housing 101 against internal pressure in the chamber 108 during operation. The tubing set 102 is arranged to pass through the end cap 109. According to some other embodiments, the end cap 109 is secured to the housing 101 of the sub-assembly 100 via mechanical fasteners, cooperating snap-fit features or another applicable process, such as ultrasonic welding.

The operational process of the sub-assembly will be described in detail now. According to some embodiments, the first medicament is provided initially in the first lumen tube 1021 and the first fluid pressure is effectuated in the chamber 108, the first medicament is delivered via the first administration path 1031, when a second medicament begins to be provided in the second lumen tube 1022 and the first medicament is stopped to be provided in the first lumen tube 1021, the second fluid pressure is effectuated in the chamber 108, the second fluid pressure is higher than the first fluid pressure. When the fluid pressure change exceeds the predetermined level, the shuttle 105 is transversally moved from the first position 1051 to the second position 1052 due to the fluid pressure change, and the first administration path 1031 is blocked and the second administration path 1032 is opened due to the transverse movement of the shuttle 105, the second medicament is delivered via the second administration path 1032.

Further according to some embodiments, the shuttle 105 comprises a first opening 1053 and a second opening 1054 as shown in FIGS. 4, 5 and 7. The first opening 1053 is arranged to provide the first medicament from the tubing set 102 to the first administration path 1031 and the second opening 1054 is arranged to provide the second medicament from the tubing set 102 to the second administration path 1032, a transverse distance between the first opening 1053 and the second opening 1054 is shorter than the distance between the first administration path 1031 and the second administration path 1032 such that the first administration path 1031 is opened and the second administration path 1032 is blocked when the shuttle 105 is in the first position 1051 and the first administration path 1031 is blocked and the second administration path 1032 is opened when the shuttle 105 is in the second position 1052, thereby the first medicament or the second medicament is selectively provided via the transverse movement of the shuttle 105.

According to some embodiments, the sub-assembly 100 further comprises a needle 103. The needle 103 is arranged in the housing 101 and connected to the first administration path 1031 and the second administration path 1032. According to some embodiments, the needle 103 is connected to the first administration path 1031 and the second administration path 1032 through a soft cannula. According to some other embodiments, the needle 103 is connected to the first administration path 1031 and the second administration path 1032 through a U-shaped tube. The needle 103 is arranged to be longitudinally movable in respect to the housing 101 between a retracted position 1033 and an extended position 1034 such that a length of the needle 103 extending outside the housing 101 is adjustable. The length of the needle 103 extending outside the housing 101 when the needle 103 is in the retracted position 1033 is shorter than the length of needle 103 extending outside the housing 101 when the needle is in the extended position 1034. According to some embodiments, the needle 103 may have the shortest length extending outside the housing 101 for medicament delivery when the needle 103 is in the retracted position 1033 and may have the longest length extending outside the housing 101 for medicament delivery when the needle 103 is in the extended position 1034.

According to some embodiments, the needle 103 is arranged in the housing 101 with an elastic element 104 and a needle retention bar 106. The elastic element 104 is longitudinally arranged along the needle 103. According to some embodiments, the elastic element 104 is attached to the housing 101 at one end 1041 and attached to a needle hub 1043 at another end 1042, the needle hub 1043 is attached to the needle 103 and longitudinally movable in respect to the housing 101 such that the tension of the elastic element 104 is changed and the length of the needle 103 extending outside the housing 101 is adjusted. Specifically, when the needle 103 is at the retracted position 1033, the elastic element 104 is stretched and when the needle 103 is at the extended position 1034, the elastic element 104 is relaxed. The elastic element 104 is any suitable element having elasticity. According to some embodiments, the elastic element 104 is an elastic band. According to some other embodiments, the elastic element 104 is a spring. According to some embodiments, the elastic element 104 may be attached to the housing 101 and the needle hub 1043 through screws. According to some other embodiments, the elastic element 104 may be attached to the housing 101 and the needle hub 1043 through pins, hooks, or use of co-molding.

According to some embodiments, the needle retention bar 106 is integrated with the shuttle 105 or is a separate component and attachable to the shuttle 105. According to some embodiments, the needle retention bar 106 is arranged to be at an inner position 1061 initially to provide a support to the needle hub 1043 when the needle 103 is in the retracted position 1033 and the elastic element 104 is stretched, the needle retention bar 106 is further arranged to transversally shift between the inner position 1061 and an outer position 1062 due to the transverse movement of the shuttle 105 such that the tension of the elastic element 104 changes and correspondingly the needle 103 moves longitudinally between the retracted position 1033 and the extended position 1034. According to some embodiments, the needle retention bar 106 comprises a support surface 1063, the support surface 1063 enables the needle retention bar 106 to be engaged with the needle hub 1043 at the inner position 1061 and to be disengaged with the needle hub 1043 at the outer position 1062. According to some other embodiments, the needle retention bar 106 comprises a curved support surface 1063 and the needle hub 1043 is configured to move along the curved support surface 1063 from the inner position 1061 to the outer position 1062.

According to some embodiments, when the second medicament is provided in the tubing set 102, especially in the second lumen tube 1022, the second fluid pressure effectuated in the chamber 108 is higher than the first fluid pressure and the shuttle 105 moves from the first position 1051 to the second position 1052 and correspondingly, the needle retention bar 106 shifts from the inner position 1061 to the outer position 1062, thereby the tension state of the elastic element 104 changes from a stretched state to a relaxed state and the needle is configured to move from the retracted position 1033 to the extended position 1034. According to some embodiments, the first administration path 1031 is for subcutaneous (SC) administration and the second administration path 1032 is for intramuscular (IM) administration.

Further according to some embodiments, the fluid pressure change value may vary depending on the medicament fluid supplied in the tubing set, in such case, the shuttle 105 may move to a position different from the first position 1051 and the second position 1052. In such case, more than two openings may be provided in the shuttle 105 to selectively open/block the first administration path 1031 and the second administration path 1032. Correspondingly, in order to provide a support to the needle hub 1043 even when the needle is at a position different from the retracted position 1033 and the extended position 1034, the needle retention bar 106 is arranged to comprise multiple support surfaces. These multiple support surfaces are offset to one another in the longitudinal direction, such that the elastic element 104 is stretched in different degrees, and correspondingly, the length of the needle 103 extending outside the housing 101 is adjustable to adapt to different insertion depth for medicament delivery.

According to some embodiments, the sub-assembly 100 may further comprise an indicator 107 indicating the administration path that is being currently used. According to some other embodiments, the indicator 107 is a visible symbol that is exposed only when the second administration path 1032 is used.

According to some other embodiments, the shuttle 105 may be a wedged shuttle. Further according to some embodiments, the wedged shuttle may comprise two interlocking wedges, wherein one of which is pushed by the second lumen tube 1022 when the second medicament is provided in the second lumen tube 1022, and the other wedge is arranged to trigger the release of the needle from the retracted position 1033 to the extended position 1034. The interlocking wedges may combine with above mentioned tubing set 102, the first administration path 1031, the second administration path 1032, the needle 103, the elastic element 104 and the needle retention bar 106. Suitable adaption may apply for the combination.

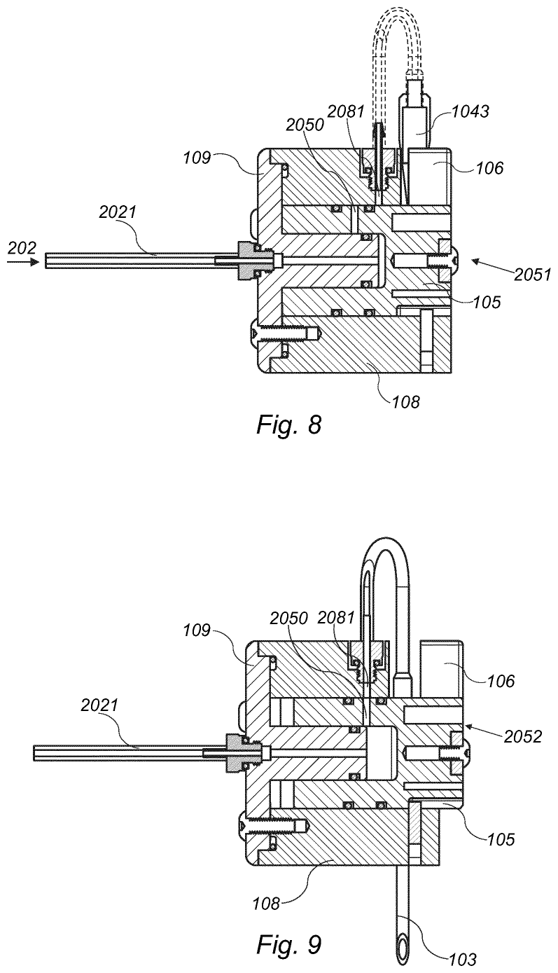

Furthermore, FIGS. 8-12 disclose a sub-assembly of the second embodiment of the invention. The sub-assembly in the second embodiment comprises basically comprises all elements as disclosed in the any of examples of the first embodiment, except that the sub-assembly in the second embodiment can be arranged with only one administration path 2031, namely, the tubing set 202 of the sub-assembly can only comprise one lumen tube 2021.

As mentioned above, the sub-assembly 200 in the second embodiment comprises the shuttle 105 arranged in the chamber 108 and is connected to a tubing set 202, which is shown in FIGS. 8, 9, and 12. The shuttle 105 is arranged to move transversally between a first position 2051 and a second position 2052 within the chamber 108, similar to the first embodiment above. The shuttle 105 is further sealably arranged in the chamber 108 and is arranged at an initial position by default. The shuttle 105 is further arranged to move from the first position 2051 to the second position 2052 when the fluid pressure change within the chamber 108 exceeds a predetermined level, such that the administration path 2031 in the chamber 108 is opened (by aligning a fluid outlet 2050 of the shuttle 105 to an outlet 2081 of the chamber 108) for delivery of medicament. Thus, as mentioned in the first embodiment, the sub-assembly 200 is activated solely through fluidic pressure without any reliance on a controller and electrical signaling, which greatly reduces the complexity of the sub-assembly. According to some embodiments, the predetermined level is a preset value. According to some other embodiments, the predetermined level is manually configurable. Furthermore, as mentioned above according to some embodiments, the end cap 109 is arranged to seal off the housing 101 against internal pressure in the chamber 108 during operation. The tubing set 202 is arranged to pass through the end cap 109. According to some other embodiments, the end cap 109 is secured to the housing 101 of the sub-assembly 200 via mechanical fasteners, cooperating snap-fit features or another applicable process, such as ultrasonic welding.

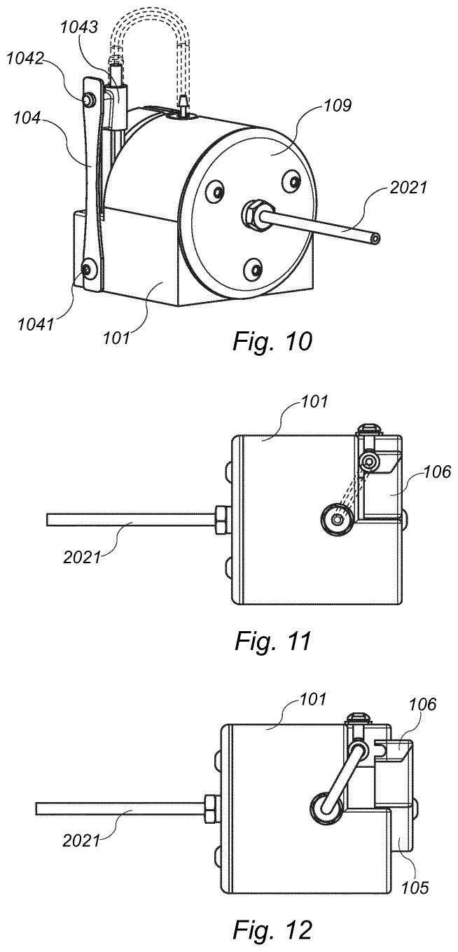

According to some embodiments, the needle 103 is arranged in the housing 101 with the elastic element 104 and the needle retention bar 106, as mentioned above in the first embodiment. The elastic element 104 is longitudinally arranged along the needle 103. According to some embodiments, the elastic element 104 is attached to the housing 101 at one end 1041 and attached to the needle hub 1043 at another end 1042, the needle hub 1043 is attached to the needle 103 and longitudinally movable in respect to the housing 101 such that the tension of the elastic element 104 is changed and the length of the needle 103 extending outside the housing 101 is adjusted, as shown in FIG. 10 for the second embodiment.

In the second embodiment, the sub-assembly 200 is configured to penetrate the skin. Thus, when the needle 103 is its initial position (the needle 103 is position within the housing 101), the elastic element 104 is stretched, as shown in FIGS. 8 and 11. When the medicament starts to flow into the chamber 108, the fluid pressure within the chamber 108 starts to accumulate. Once the accumulated fluid pressure is great enough to move the shuttle 105 from the first position 2051 to the second position 2052 so that the outlet 2081 of the chamber 108 is aligned with the outlet 2050 of the shuttle 105 to open the administration path 2031, the medicament is delivered via the administration path 2031 to the needle 103, as shown in FIG. 9. In the meantime, as the shuttle 105 is moved to the second position 2052, the needle hub 1043 is no longer engaged with the retention bar 106, as a result, the elastic element 104 is relaxed and the needle 103 is moved out of the housing to penetrate the skin, as shown in FIGS. 9 and 12.

As mentioned above in the first embodiment, the elastic element 104 is any suitable element having elasticity. According to some embodiments, the elastic element 104 is an elastic band. According to some other embodiments, the elastic element 104 is a spring. According to some embodiments, the elastic element 104 may be attached to the housing 101 and the needle hub 1043 through screws. According to some other embodiments, the elastic element 104 may be attached to the housing 101 and the needle hub 1043 through pins, hooks, or use of co-molding.

The sub-assembly 200 as disclosed in the second embodiment can be used solely. Alternatively, according to a third embodiment of the invention, the sub-assembly 100 according to the first embodiment can be used with the sub-assembly 200 according to the second embodiment. In the third embodiment, the retention bar comprises multiple support surfaces offset to one another in the longitudinal direction. In a preferred example, the retention bar comprises a staired-structured with at least two steps providing at least two support surfaces respectively, in this example, the needle 103 is in the initial position before the medicament delivery starts. The shuttle is in a first position as disclosed in the second embodiment and the needle hub is engaged with a first step of the retention bar. When the medicament delivery starts, the medicament flows into the chamber, as a result, the fluid pressure moves the shuttle to the second position as disclosed in the second embodiment. The first step of the retention bar is disengaged from the needle hub; therefore, the needle is moved to penetrate the skin under the force of the elastic element. When the needle is in the retracted position as disclosed in the first embodiment, the needle hub is engaged with a second step of the retention bar. Furthermore, when a second medicament is delivered, the fluid pressure can move the shuttle further as disclosed in the first embodiment. As a result, the second step of the retention bar is moved away from the needle hub together with the shuttle; therefore, the needle can be moved to the extended position under the force of the elastic element.

Furthermore, manipulation of the housing, needle hub, retention bar, and internal fluidics may all be used to modify the desired extension of the needle, ether for the initial penetration depth or for the further depth to deliver the second medicament. Similarly, adjustments to the elastic band, internal chamber dimensions, and frictional/clearance between components may be used to adjust the sensitivity of the release mechanism.

The concept as shown has a direct connection to the tubing set. However, tubing may be connected to a fluidic connector on the needle assembly (e.g., barbed, Luer, or other suitable connector). This may be done by a user, dispensing pharmacists, or during manufacturing. This is advantageous for manufacturing or when the fluid reservoir has an integrated tubing set, and the needle is attached to the tubing set after the reservoir is filled. It allows multiple needle assemblies (i.e., needle gauges or insertion depths) to be provided (e.g., as a kit of components) and selected based on patient anatomy or drug characteristics.

Importantly, this invention allows the needle depth to be controlled to any SC length required. For instance, larger infusions might require longer needles (e.g., 12 mm) while shorter needles (e.g., 4-6 mm) may suffice for smaller infusions. The needle hub may be adapted (e.g., separate molded components with lengthened/shortened hub) to allow various insertion depths, requiring no change to the outer housing, shuttle, or other components. Alternatively, the needle stop (e.g., retention bar) could be adapted to allow multiple depths to be selected during use, allowing a single assembly to provide a full range of insertion depths.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-la′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), immunoglobulins (primary immune deficiencies), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab, diphenhydramine, acetaminophen, ondansetron, famotidine, epinephrine, naloxone, hydrocortisone, dexamethasone, or methylprednisolone.

Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims.

1-15. (canceled)

16. A sub-assembly for a medicament delivery device, comprising:
a housing;
a closed chamber within the housing;
a tubing set transversally connected to the housing, wherein the tubing set is arranged to provide a medicament flow, a fluid pressure is effectuated in the chamber when the medicament is provided; and
a shuttle arranged in the chamber and connected to the tubing set, wherein the shuttle is configured to move transversally between a first position and a second position within the chamber when the fluid pressure exceeds a predetermined level, such that an administration path in the chamber is opened for delivery of the medicament.

17. The sub-assembly for a medicament delivery device according to claim 16, wherein the tubing set is arranged to provide one of a first medicament flow and a second medicament flow, a first fluid pressure is effectuated in the chamber when the first medicament is provided and a second fluid pressure having a different value from the first fluid pressure is effectuated in the chamber when the second medicament is provided, thereby a fluid pressure change is effectuate due to a change between the provision of the first medicament and the second medicament, and wherein the shuttle is configured to move transversally between a first position and a second position within the chamber when the fluid pressure change exceeds a predetermined level, such that a first administration path or a second administration path in the chamber is opened for selectively delivery of the first medicament and the second medicament.

18. The sub-assembly for a medicament delivery device according to claim 16, further comprising:
a needle connected to the administration path and arranged in the housing, wherein the needle is arranged to be longitudinally movable in respect to the housing when the shuttle is move transversally from the first position to the second position.

19. The sub-assembly for a medicament delivery device according to claim 18, wherein the needle is connected to the first administration path and the second administration path, wherein the needle is arranged to be longitudinally movable in respect to the housing between a retracted position and an extended position such that a length of the needle extending outside the housing is adjustable, wherein the length of the needle extending outside the housing when the needle is in the retracted position is shorter than the length of needle extending outside the housing when the needle is in the extended position.

20. The sub-assembly for a medicament delivery device according to claim 18, further comprising:
an elastic element longitudinally arranged along the needle and attached to the housing at one end and attached to a needle hub at another end, wherein the needle hub is attached to the needle and longitudinally movable in respect to the housing such that the tension of the elastic element is changed and the length of the needle extending outside the housing is adjusted, and
a needle retention bar arranged to releasably engage with the needle hub when the needle,
wherein the needle retention bar is further arranged to transversally shift between an inner position and an outer position due to the transverse movement of the shuttle such that the needle moves longitudinally in respect to the housing.

21. The sub-assembly for a medicament delivery device according to claim 20, wherein the needle retention bar engaged with the needle hub when the needle is in the retracted position, and the needle retention bar is further arranged to transversally shift between an inner position and an outer position due to the transverse movement of the shuttle such that the needle moves longitudinally between the retracted position and the extended position.

22. The sub-assembly for a medicament delivery device according to claim 21, wherein the needle is arranged to extend outside the housing from the retracted position to the extended position when the second fluid pressure is effectuated in the chamber by providing the second medicament in the tubing set, wherein the second fluid pressure is higher than the first fluid pressure.

23. The sub-assembly for a medicament delivery device according to claim 20, wherein the shuttle is configured to move from the first position to the second position and correspondingly, the needle retention bar is configured to shift from the inner position to the outer position, thereby the tension of the elastic element changes and the needle is moved in respect of the housing.

24. The sub-assembly for a medicament delivery device according to claim 17, wherein the tubing set comprises:
a first lumen tube for the first medicament flow, and
a second lumen tube for the second medicament flow.

25. The sub-assembly for a medicament delivery device according to claim 24, wherein the second lumen tube has a smaller diameter than the first lumen tube.

26. The sub-assembly for a medicament delivery device according to claim 17, wherein the shuttle comprises a first opening for providing the first medicament from the tubing set to the first administration path and a second opening for providing the second medicament from the tubing set to the second administration path, wherein a transverse distance between the first opening and the second opening is shorter than the distance between the first administration path and the second administration path thereby the first administration path is opened and the second administration path is blocked when the shuttle is in the first position and the second administration path is opened and the first administration path is blocked when the shuttle is in the second position such that the first medicament or the second medicament is selectively provided via the transverse movement of the shuttle.

27. The sub-assembly for a medicament delivery device according to claim 20, wherein the needle retention bar is integrated with the shuttle or the needle retention bar is a separate component attached to the shuttle.

28. The sub-assembly for a medicament delivery device according to claim 20, wherein the needle retention bar comprises multiple support surfaces offset to one another in the longitudinal direction.

29. The sub-assembly for a medicament delivery device according to claim 18, wherein the needle is arranged to be longitudinally movable in respect to the housing when the shuttle is move transversally from the first position to the second position such that the needle is moved out of the housing.

30. The sub-assembly for a medicament delivery device according to claim 20, wherein the needle is moved out of the housing when the shuttle is move transversally from the first position to the second position by the elastic element.
